(19) United States
(12) Patent Application Publication
(10) Pub. No.: US 2015/0094566 A1
(43) Pub. Date: Apr. 2, 2015

(54) METHOD OF CONTROLLING ROUTE OF ANGIOCATHETER USING OPTICAL COHERENCE TOMOGRAPHY AND ANGIOGRAPHY APPARATUS FOR PERFORMING THE SAME

(71) Applicant: Samsung Electronics Co., Ltd., Suwon-si (KR)

(72) Inventors: Ji-won RYU, Suwon-si (KR); Jae-guyn LIM, Seongnam-si (KR); Woo-young JANG, Seongnam-si (KR); Jae-duck JANG, Suwon-si (KR)

(73) Assignee: SAMSUNG ELECTRONICS CO., LTD., Suwon-si (KR)

(21) Appl. No.: 14/501,335

(22) Filed: Sep. 30, 2014

(30) Foreign Application Priority Data
Sep. 30, 2013 (KR) 10-2013-0116459

(51) Int. Cl. A61B 5/00; A61K 49/04; A61B 6/00; A61M 5/00; A61B 19/00; A61M 25/01

(52) U.S. Cl. CPC A61B 5/0035; A61B 19/5244; A61B 5/0066; A61M 25/0105; A61B 6/481; A61M 5/007; A61B 6/461; A61K 49/04; A61B 5/742; A61B 5/489; A61B 6/504; A61B 2019/5234; A61B 2019/5238; A61B 2576/02; A61M 2210/12; A61B 2019/5255
USPC 600/424

(57) ABSTRACT

Provided is a method of controlling an angiocatheter route using OCT. The method includes inserting a catheter into a blood vessel of a test object, injecting a dye into the blood vessel and capturing an X-ray image, acquiring a three-dimensional OCT image of a vicinity around the catheter, determining a position of the catheter within the blood vessel using the three-dimensional OCT image and the X-ray image, and displaying the position of the catheter on the X-ray image.

### CROSS-REFERENCE TO RELATED APPLICATION

This application claims priority from Korean Patent Application No. 10-2013-0116459, filed on Sep. 30, 2013, in the Korean Intellectual Property Office, the disclosure of which is incorporated herein by reference in its entirety.

### BACKGROUND

1. Field

Apparatuses and methods consistent with exemplary embodiments relate to a method of controlling a route of an angiocatheter when performing angiography using optical coherence tomography, and an angiography apparatus for performing the method.

2. Description of the Related Art

Angiography is a method of inserting a catheter into a blood vessel of a test object, injecting a dye through which X-rays hardly transmits, and capturing an image from the X-rays of the blood vessels which may be used to diagnose a condition of the blood vessels, such as for diagnosing an abnormality of the blood vessels.

However, because X-ray imaging is performed in real time, a test object may be excessively exposed by radiation, and the amount of dye to be injected may be large. In addition, it may be difficult to determine the depth or width of a blood vessel through an X-ray image acquired by capturing a blood vessel into which a dye is injected, and to ascertain an exact position of a lesion within the blood vessel. For example, it may be difficult to ascertain the position in an up, down, left, and right direction of the lesion.

Optical coherence tomography may be capable of imaging an internal structure of an object using interference between light, with which the object is irradiated and which is reflected, and reference light has been widely used in a medical field because the optical coherence tomography allows a high-resolution image to be acquired and is harmless to humans.

### SUMMARY

According to an aspect of an exemplary embodiment, there is provided a method of imaging a catheter using optical coherence tomography (OCT), the method including inserting a catheter into a blood vessel of a test object, injecting a dye into the blood vessel and capturing an X-ray image, acquiring a three-dimensional OCT image of a vicinity around the catheter, determining a position of the catheter within the blood vessel using the three-dimensional OCT image and the X-ray image, and displaying the position of the catheter on the X-ray image.

The method may further include controlling a movement route through the blood vessel of the catheter on the basis of the displayed position.

The determining of the position of the catheter may include matching the three-dimensional OCT image and the X-ray image with each other by comparing shapes of the blood vessels shown in the three-dimensional OCT image with shapes of the blood vessels shown in the X-ray image, and determining a position of the catheter in the blood vessel shown in the X-ray image based on the position of the catheter in the three-dimensional OCT image.

The matching of the three-dimensional OCT image and the X-ray image may include matching the three-dimensional OCT image and the X-ray image by comparing boundary patterns of the blood vessels shown in the three-dimensional OCT image and the X-ray image.

The matching of the three-dimensional OCT image and the X-ray image may include matching the three-dimensional OCT image and the X-ray image by comparing gradients and the degrees of bending of the blood vessels shown in the three-dimensional OCT image and the X-ray image.

The matching of the three-dimensional OCT image and the X-ray image may include matching the three-dimensional OCT image and the X-ray image by segmenting the blood vessels shown in the three-dimensional OCT image and the X-ray image into a plurality of portions and then comparing the segmented portions with each other.

The matching of the three-dimensional OCT image and the X-ray image may include matching the three-dimensional OCT image and the X-ray image by segmenting the blood vessels shown in the three-dimensional OCT image and the X-ray image into a plurality of portions and then comparing the segmented portions with each other.

The three-dimensional OCT image is a blood vessel image that may be constituted by at least one of a three-dimensional OCT image taken continuously over time and an OCT image showing a cross-section of the blood vessel image taken in one direction.

The method may further include determining a position and a state of a lesion within the blood vessel using the three-dimensional OCT image, and displaying the position and the state of the lesion on the X-ray image.

The method may further include injecting a dye into a region of the blood vessel in which the catheter is placed and capturing another X-ray image in response to the catheter moving out of the region of the X-ray image.

The method may further include measuring a movement distance and a movement time of the catheter, and calculating a movement speed of the catheter based on the measured movement distance and movement time.

The displaying of the position of the catheter may include displaying the three-dimensional OCT image at the determined position of the catheter.

A non-transitory computer readable medium on which are stored computer instructions and data that, when executed by a computer, execute the method.

According to an aspect of another exemplary embodiment, there is provided an angiography apparatus including a catheter configured to be inserted into a blood vessel of a test object, an optical probe configured to capture a three-dimensional optical coherence tomography (OCT) image of a vicinity around the catheter, an X-ray image acquisition unit configured to acquire an X-ray image of the blood vessel of the test object into which a dye is injected, a three-dimensional OCT image acquisition unit configured to receive the three-dimensional OCT image from the optical probe on the catheter, a catheter position ascertainment unit configured to determine a position of the catheter within the blood vessel using the X-ray image acquired by the X-ray image acquisition unit and the three-dimensional OCT image acquired by the three-dimensional OCT image acquisition unit, and an image display unit configured to display the ascertained position of the catheter on the X-ray image.

The catheter position ascertainment unit may include an image matching unit configured to match the three-dimensional OCT image and the X-ray image with each other by comparing shapes of the blood vessels shown in the three-dimensional OCT image with shapes of the blood vessels shown in the X-ray image, and a position correspondence unit configured to determine a position of the catheter in the blood vessel shown in the X-ray image based on the position of the catheter in the three-dimensional OCT image.

The image matching unit may be further configured to match the three-dimensional OCT image and the X-ray image by comparing boundary patterns of the blood vessels shown in the three-dimensional OCT image and the X-ray image.

The image matching unit may be further configured to match the three-dimensional OCT image and the X-ray image by comparing gradients and the degrees of bending of the blood vessels shown in the three-dimensional OCT image and the X-ray image.

The image matching unit may be further configured to match the three-dimensional OCT image and the X-ray image by segmenting the blood vessels shown in the three-dimensional OCT image and the X-ray image into a plurality of portions and then compare the segmented portions with each other.

The image matching unit may be further configured to match the three-dimensional OCT image and the X-ray image by segmenting the blood vessels shown in the three-dimensional OCT image and the X-ray image into a plurality of portions and then compare the segmented portions with each other.

The three-dimensional OCT image may be a blood vessel image constituted by at least one of a three-dimensional OCT image taken continuously over time and an OCT image showing a cross-section of the blood vessel image taken in one direction.

The angiography apparatus may further include an image analysis unit configured to determine a position and a state of a lesion within the blood vessel using the three-dimensional OCT image acquired by the three-dimensional OCT image acquisition unit, wherein the image display unit is configured to display the position and the state of the lesion on the X-ray image.

The X-ray image acquisition unit may be further configured to acquire another X-ray image of the region in which the catheter is placed in response to the catheter moving out of a region of the X-ray image.

The image display unit may display the three-dimensional OCT image received by the three-dimensional OCT image acquisition unit at the determined position of the catheter.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 is a diagram illustrating a configuration of an angiography apparatus according to an exemplary embodiment;

FIG. 2 is a diagram illustrating a catheter of a angiography apparatus being inserted into a blood vessel according to an exemplary embodiment;

FIG. 3 is a diagram illustrating an X-ray image acquired by an angiography apparatus according to an exemplary embodiment;

FIG. 4 is a diagram illustrating a three-dimensional optical coherence tomography (OCT) image acquired by an angiography apparatus according to an exemplary embodiment;

FIGS. 5 through 7 are diagrams illustrating images output from an angiography apparatus according to one or more exemplary embodiments; and

FIGS. 8 through 10 are flowcharts illustrating methods of controlling a route of an angiocatheter using optical coherence tomography according to one or more exemplary embodiments.

### DETAILED DESCRIPTION OF THE DRAWINGS

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects of the present description.

The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. The progression of processing steps and/or operations described is an example; however, the sequence of and/or operations is not limited to that set forth herein and may be changed as is known in the art, with the exception of steps and/or operations necessarily occurring in a particular order. In addition, respective descriptions of well-known functions and constructions may be omitted for increased clarity and conciseness.

Additionally, exemplary embodiments will now be described more fully hereinafter with reference to the accompanying drawings. The exemplary embodiments may, however, be embodied in many different forms and should not be construed as being limited to the embodiments set forth herein. These embodiments are provided so that this disclosure will be thorough and complete and will fully convey the exemplary embodiments to those of ordinary skill in the art. The scope is defined not by the detailed description but by the appended claims. Like numerals denote like elements throughout.

The term “ . . . unit” used in the embodiments indicates a component including software or hardware, such as a Field Programmable Gate Array (FPGA) or an Application-Specific Integrated Circuit (ASIC), and the “ . . . unit” performs certain roles. However, the “ . . . unit” is not limited to software or hardware. The “ . . . unit” may be configured to be included in an addressable storage medium or to reproduce one or more processors. Therefore, for example, the “ . . . unit” includes components, such as software components, object-oriented software components, class components, and task components, processes, functions, attributes, procedures, subroutines, segments of program code, drivers, firmware, microcode, circuits, data, a database, data structures, tables, arrays, and variables. A function provided inside components and “ . . . units” may be combined into a smaller number of components and “ . . . units”, or further divided into additional components and “ . . . units”.

According to one or more exemplary embodiments, optical coherence tomography may be combined with angiography, and thus a moving route of an angiocatheter may be controlled only using a relatively small number of times of X-ray imaging, and a lesion may be exactly diagnosed.

FIG. 1 is a diagram illustrating a configuration of an angiography apparatus according to an exemplary embodiment of the present disclosure. FIG. 1 illustrates only components related to the exemplary embodiment of the present disclosure, and thus the angiography apparatus may further include other general components.

Referring to FIG. 1, the angiography apparatus according to an exemplary embodiment may include a catheter 10, an optical probe 21, a dye supply unit 40, and a main body 100. The catheter 10 may be connected to the main body 100 through a cable 30, and the optical probe 21 may be connected to the main body 100 through an optical fiber 20.

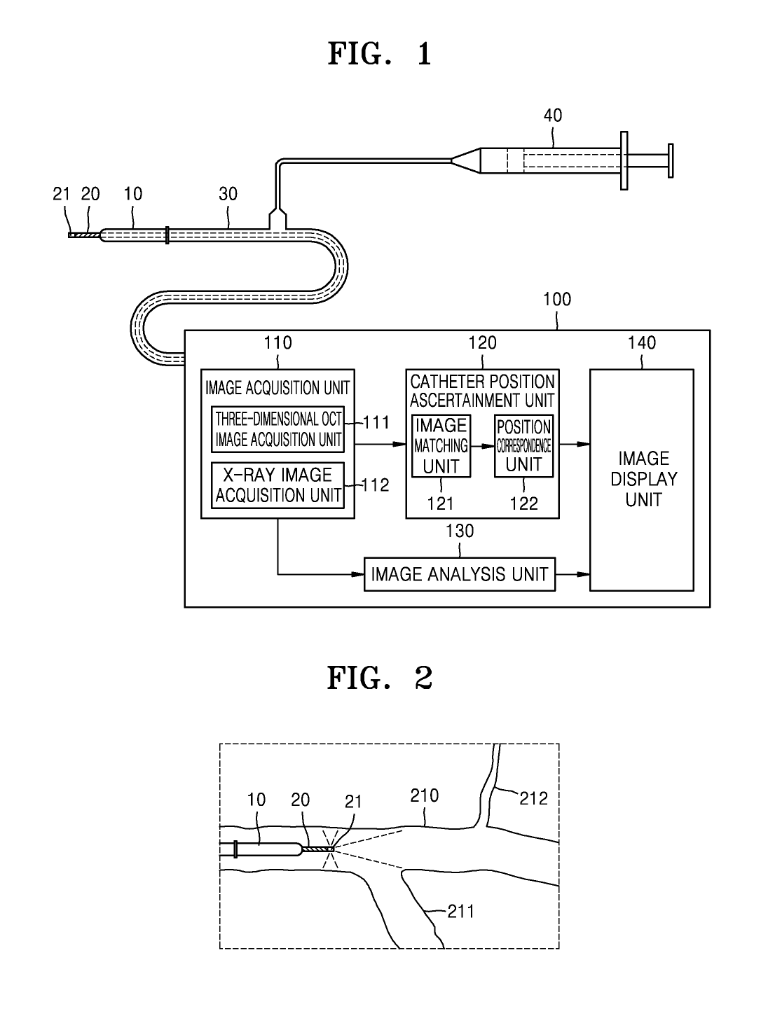

The main body 100 may include an image acquisition unit 110, a catheter position ascertainment unit 120, an image analysis unit 130, and an image display unit 140. Among these, the image acquisition unit 110 may include a three-dimensional optical coherence tomography (hereinafter, referred to as OCT) image acquisition unit 111 and an X-ray image acquisition unit 112, and the catheter position ascertainment unit 120 may include an image matching unit 121 and a position correspondence unit 122. A detailed operation of each of the components of the main body 100 will be described below with reference to FIGS. 2 through 7.

The catheter 10 may have a diameter of approximately 1 mm to 2 mm, and may be inserted into a blood vessel of a test object. The catheter 10 may be inserted into the blood vessel and then moved to a region of interest (ROI). When the catheter 10 is located in the region of interest, a dye supplied from the dye supply unit 40 may be injected into the blood vessel of the region of interest through the catheter 10, and then X-ray imaging may be performed. A material hardly transmitting X-rays, for example, a barium suspension or an iodine preparation may be used as the dye.

When the X-ray imaging is performed in a state where the dye is injected into the blood vessel, an X-ray image capable of distinguishing the blood vessel may be acquired. The X-ray image acquired in this manner may be analyzed so as to determine whether blood vessel occlusion occurs or whether a lesion is present within the blood vessel.

However, X-ray imaging is performed in real time in order to ascertain the position of the catheter 10 while the catheter 10 moves along a blood vessel. Accordingly, a test object may be excessively exposed to radioactivity, and an excessive amount of dye may be injected into a blood vessel due to a continuous imaging operation. In addition, it is difficult to determine the depth or width of a blood vessel and to ascertain an exact position of a lesion within the blood vessel using only an X-ray image.

Therefore, in an exemplary embodiment an OCT function may be combined with the angiography apparatus.

The optical probe 21 is a component that may be used to perform OCT to acquire an OCT image. OCT is a technique that irradiates an object with measurement light and images an internal structure of the object using an interference phenomenon between the measurement light reflected from the object and reference light. That is, the optical probe 21 may irradiate the inside of a blood vessel with light to acquire an image of the internal structure of the blood vessel.

The optical probe 21 may be manufactured integrally with the catheter 10, or may be manufactured integrally with a guide wire which guides a moving route of the catheter 10. Alternatively, the optical probe 21 may be manufactured to be separated from the catheter 10 or the guide wire.

The optical probe 21 may always irradiate light within the inside of the blood vessel in the vicinity of the catheter 10 to acquire an OCT image of the area in the vicinity of the catheter 10, regardless of whether the optical probe 21 is manufactured integrally with the catheter 10 or the guide wire. In the current exemplary embodiment, the optical probe 21 may acquire a three-dimensional OCT image, but the scope of the present disclosure is not limited thereto.

In this manner, the three-dimensional OCT image of the vicinity of the catheter 10 may be acquired using the optical probe 21, and thus it is possible not only to control the moving route of the catheter 10 in real time, but also to ascertain an exact position and state of a lesion from the OCT image.

Hereinafter, an operation of the angiography apparatus according to an exemplary embodiment will be described in detail with reference to FIGS. 2 through 7.

FIG. 2 is a diagram illustrating a state where the catheter 10 of the angiography apparatus, according to an exemplary embodiment, is inserted into a blood vessel. Referring to FIG. 2, the catheter 10 is inserted into a vessel lumen 210. The optical probe 21 coupled to the catheter 10 irradiates the inside of the blood vessel with light. The three-dimensional OCT image acquisition unit 111 included in the main body 100 may acquire a three-dimensional OCT image for the inside of the blood vessel through the optical probe 21. When the three-dimensional OCT image acquisition unit 111 acquires the three-dimensional OCT image for the inside of the blood vessel, the image analysis unit 130 may analyze the three-dimensional OCT image to ascertain the position and state of a lesion within the blood vessel, and may ascertain the structure of the blood vessel. For example, as illustrated in FIG. 2, structures of side branch blood vessels 211 and 212 may be ascertained.

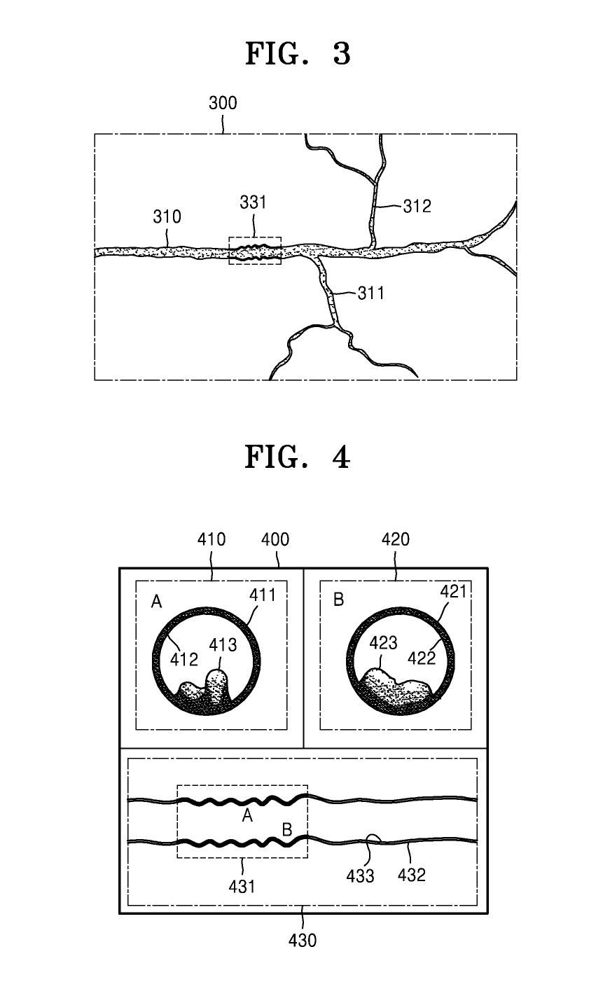

The catheter 10 may move within and along a blood vessel. When doing so it may be possible to exactly ascertain the structure of the blood vessel and the position of the catheter 10 within the blood vessel in order to reduce the damage of the blood vessel as much as possible and to move the catheter 10 to a region of interest. Accordingly, when the catheter 10 is inserted into the blood vessel, a dye is injected into the blood vessel, and then X-ray imaging is performed. An image acquired by injecting the dye into the blood vessel and then performing the X-ray imaging is illustrated in FIG. 3.

FIG. 3 is a diagram illustrating an X-ray image acquired by the angiography apparatus according to an exemplary embodiment. That is, the X-ray image is an image acquired by an X-ray image acquisition unit 112 as shown in FIG. 1. Referring to FIG. 3, in an X-ray image 300, blood vessels 310, 311, and 312 into which a dye is injected are shown to be distinguishable. A structure of the blood vessel may be ascertained through such an X-ray image. However, according to the current exemplary embodiment of the present disclosure, the X-ray imaging is performed once at the initial stage when the catheter 10 is inserted into the blood vessel and is then performed again after the elapse of a substantial amount of time, and thus a three-dimensional OCT image has to be used in order to ascertain the position of the catheter 10 while moving within the blood vessel. A method of ascertaining the position of the catheter 10 using a three-dimensional OCT image will be described below in detail with reference to FIG. 4.

FIG. 4 is a diagram illustrating a three-dimensional OCT image obtained by the angiography apparatus according to an exemplary embodiment. The three-dimensional OCT image may be an image obtained by an three-dimensional OCT image acquisition unit 111 as shown in FIG. 1. Referring to FIG. 4, a three-dimensional OCT image 400 includes images 410 and 420 of a cross-section in a thickness direction of a blood vessel and an image 430 of a cross-section in a longitudinal direction of the blood vessel.

First, in the image 410 of the cross-section in the thickness direction of the blood vessel, an outer wall 411 and an inner wall 412 of the blood vessel may be distinguished from each other, and a lesion 413 may be ascertained. Similarly, in the image 420 along the cross-section in the thickness direction of the blood vessel, an outer wall 421 and an inner wall 422 of the blood vessel may be distinguished from each other, and a lesion 423 may be ascertained. Characters A and B shown in each image are for ascertaining the position of each image in the image 430 of the cross-section in the longitudinal direction of the blood vessel.

In the image 430 of the cross-section in the longitudinal direction of the blood vessel, an outer wall 432 and an inner wall 433 of the blood vessel may be ascertained, and characters indicating the imaging position of the two images 410 and 420 are shown.

In this manner, the position and state of a lesion within a blood vessel may be diagnosed more quickly and precisely by using the three-dimensional OCT image 400 of the inside of the blood vessel.

The catheter position ascertainment unit 120 of FIG. 1 may ascertain the position of the catheter 10 using the three-dimensional OCT image 400 as shown in FIG. 4 which is acquired by the three-dimensional OCT image acquisition unit 111 and the X-ray image 300 of FIG. 3 which is acquired by the X-ray image acquisition unit 112. In detail, the image matching unit 121 of the catheter position ascertainment unit 120 compares the shapes of the blood vessels shown in the three-dimensional OCT image 400 and the X-ray image 300, respectively, to match the two images with each other. In other words, the image matching unit 121 searches for a region in the X-ray image 300 that corresponds to the blood vessel shown in the three-dimensional OCT image 400.

According to an exemplary embodiment, the image matching unit 121 may search for, in the X-ray image 300, a portion which is consistent with a boundary pattern of the blood vessel in a partial region 431 of the blood vessel shown in the three-dimensional OCT image 400. Referring to FIG. 3, a region 331 consistent with the boundary pattern of the blood vessel shown in the three-dimensional OCT image 400 is illustrated.

When the two images are matched with each other by the image matching unit 121, the position correspondence unit 122 of the catheter position ascertainment unit 120 determines a position of the catheter in the blood vessel shown in the X-ray image 300 based on the position of the catheter in the three dimensional OCT image 400. Specifically, when the catheter 10 is located in the region 431 of the three-dimensional OCT image 400 of FIG. 4, the position of the catheter 10 is determined to be in the region 331 of the X-ray image 300 of FIG. 3 based on the corresponding matching of the two images.

The image matching unit 121 may use not only a method of comparing boundary patterns of blood vessels with each other but may also use any of various other methods such as a method of comparing characteristics of the blood vessel such as a gradient or the degree of bending of the blood vessel.

In addition, the image matching unit 121 may segment the blood vessels shown in the two images into a plurality of portions, compare the segmented portions with each other, and then match the two images with each other.

In this manner, the catheter position ascertainment unit 120 may ascertain the position of the catheter 10, and the image analysis unit 130 may ascertain the position and state of a lesion within a blood vessel, and then the image display unit 140 may display the ascertained content on an X-ray image and output the X-ray image.

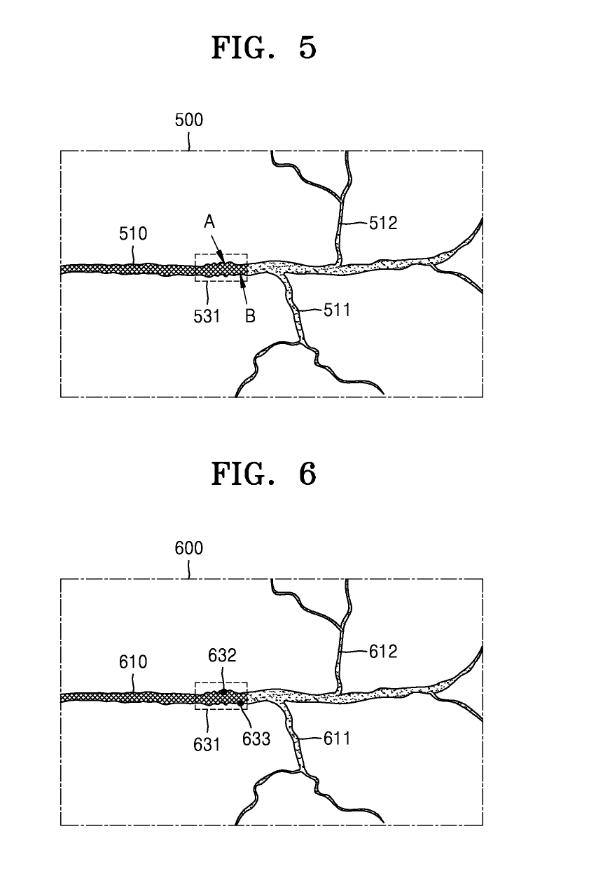

FIGS. 5 through 7 are diagrams illustrating images that are output by the angiography apparatus according to one or more exemplary embodiments.

Referring to FIG. 5, an image display unit 140 as shown in FIG. 1 may display a region 531 into which the catheter 10 is currently inserted in a blood vessel shown in an X-ray image 500 using a different color from other parts of the blood vessel. That is, the X-ray image 500 of FIG. 5 shows that the catheter 10 is inserted up to a portion shown by a dark color.

The position of the catheter 10 may be shown in a color, and the position of a lesion which is ascertained by the image analysis unit 130 may be shown by a character. For example, characters A and B shown in FIG. 5 indicate positions of lesions ascertained in the three-dimensional OCT image 400 of FIG. 4, respectively.

Referring to FIG. 6, the image display unit 140 may display a region 631 into which the catheter 10 is currently inserted in a blood vessel shown in an X-ray image 600 in a different color from other parts of the blood vessel. That is, the X-ray image 600 of FIG. 6 shows that the catheter 10 is inserted up to a portion which is currently shown in a dark color.

The position of the catheter 10 may be shown in a color, and the position of a lesion which is ascertained by the image analysis unit 130 may be shown by a character. That is, points 632 and 633 correspond to the respective lesions in different colors depending on the vertical position of the lesion. The horizontal position of the lesion in the X-ray image 600 which is a two-dimensional image, may be ascertained by the positions of the points 632 and 633, whereas the vertical position thereof may not be ascertained. Thus, an exact position of a lesion may be shown a lesion, for example, using a method of indicating a lesion located on the upper side in red and indicating a lesion located on the lower side in blue. Alternatively, other visual representation may be implemented to indicate to a viewer that the lesion is on the upper or lower side of the blood vessel. For example, a small up and down arrow graphic may be used to indicate the upper and lower sides, respectively. Another example would be providing the upper indicator in a solid visual form while the lower visual form may be provided such that it appears partially transparent or translucent given the appears to a viewer that the indicator is on the bottom surface away from the user's point of reference when looking at the image. Other visual options may exists as well which would provide an indication of where the lesion is located.

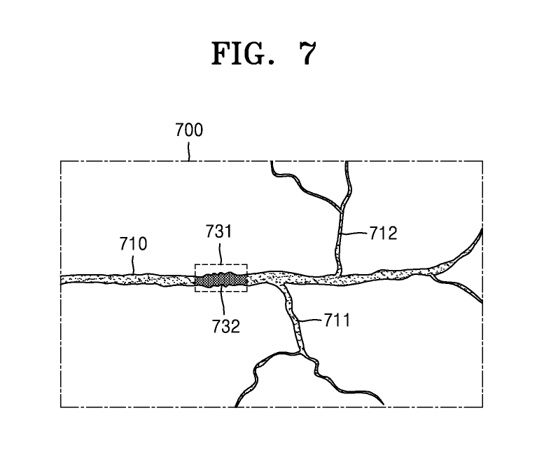

Referring to FIG. 7, the image display unit 140 may display an OCT image for a cross-section in a longitudinal direction of a blood vessel shown in an X-ray image 700, among OCT images acquired by the three-dimensional OCT image acquisition unit 111, in a region 731 in which the catheter 10 is currently located in the blood vessel. In this manner, a real OCT image 732 may be overlaid with the region 731 corresponding to the current position of the catheter 10 in the X-ray image 700 so as to display the position of the catheter 10 and to provide a precise OCT image for the corresponding position.

When the catheter 10 moves out of a region of an acquired X-ray image, a dye is injected into a region in which the catheter 10 is placed, and the X-ray image acquisition unit 112 may newly acquire an X-ray image for the region. The ascertainment of the position of the catheter 10 and the position and state of a lesion in the newly acquired X-ray image may be performed in a similar manner to the above-described method.

In addition, a distance moved by the catheter 10 may be measured using a mechanical rotary actuator that controls the movement of the catheter 10, and a movement speed of the catheter 10 may be calculated by measuring time required for the movement of the catheter 10, and thus a moving route of the catheter 10 may be effectively controlled.

In this manner, a three-dimensional OCT image for the inside of a blood vessel around a catheter may be captured, and the captured three-dimensional OCT image is matched with an X-ray image of the blood vessel, and thus the position of the catheter within the blood vessel may be ascertained. Accordingly, X-ray imaging is performed on a region of interest only once, and then a moving route of the catheter is controlled while capturing the three-dimensional OCT image, and this may be displayed on the captured X-ray image.

In addition, an exact position of the lesion may be ascertained using the captured three-dimensional OCT image, and this may be displayed on an X-ray image.

Furthermore, positional information of the catheter and tissue information of the blood vessel are acquired in real time, and the moving route of the catheter may be controlled on the basis of the pieces of information, thereby preventing a wall or tissues of the blood vessel or tissues from being damaged.

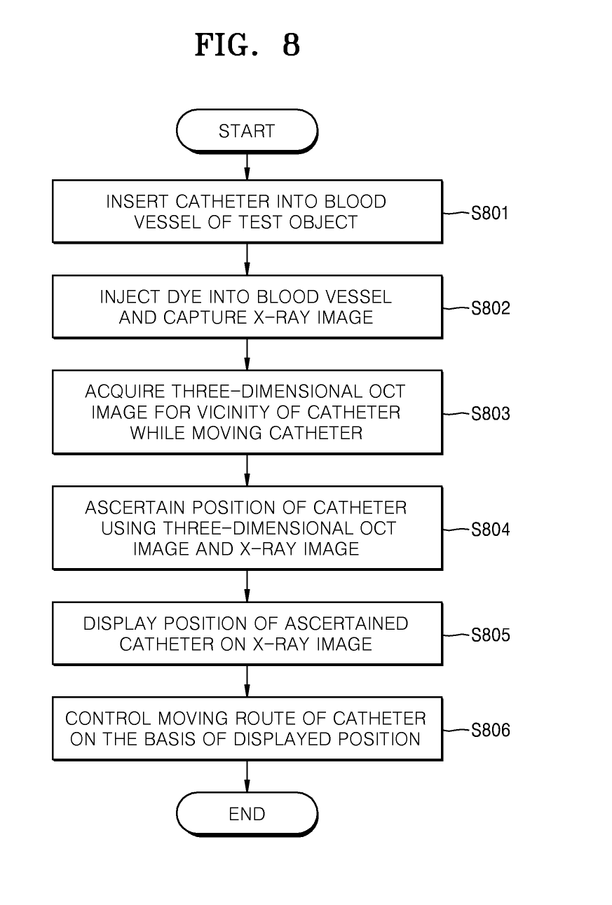

FIGS. 8 to 10 are flow charts illustrating a method of controlling a route of an angiocatheter using the OCT according to exemplary embodiments of the present disclosure.

Referring to FIG. 8, in operation S801, a catheter is inserted into a blood vessel of a test object. When the catheter is inserted into the blood vessel, a dye is injected into the blood vessel through the catheter and an X-ray image for the blood vessel is captured in operation S802. The structure of the blood vessel may be ascertained from the captured X-ray image. In operation S803, a three-dimensional OCT image for the vicinity of the catheter is acquired while moving the catheter. In detail, an OCT image for a cross-section of the blood vessel in the thickness direction and an OCT image for a cross-section of the blood vessel in the longitudinal direction may be acquired. An example of the acquired three-dimensional OCT image is illustrated in FIG. 4.

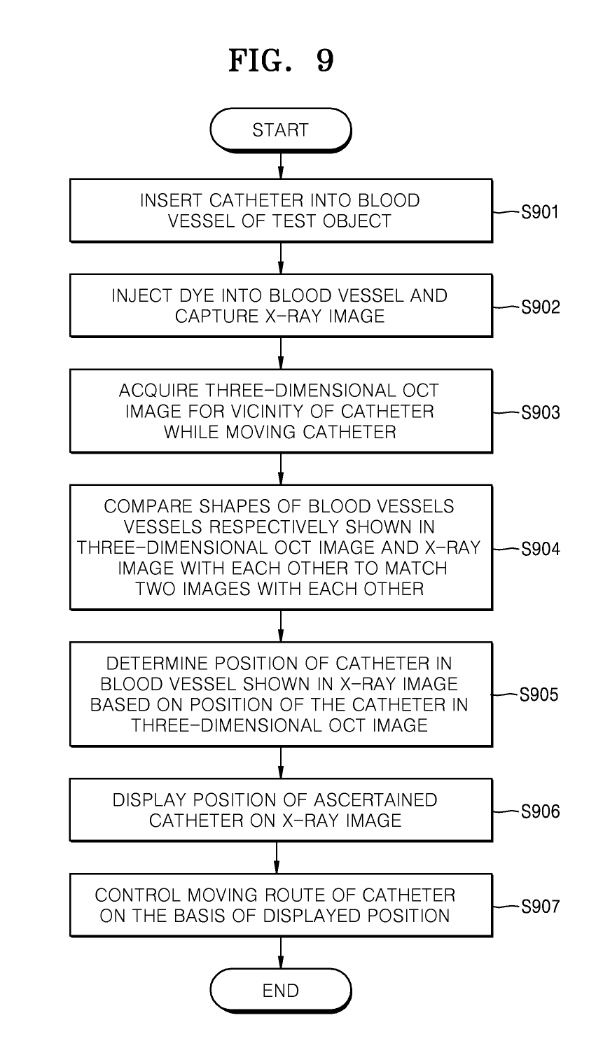

When both the X-ray image and the three-dimensional OCT image are acquired, the position of the catheter is ascertained using the X-ray image and the three-dimensional OCT image in operation S804. A description of the ascertainment of the position of the catheter using the two images will be given later in detail with reference to FIG. 9. When the position of the catheter is ascertained, in operation S805, the ascertained position of the catheter is displayed on the X-ray image. Finally, in operation S806, a subsequent moving route of the catheter is controlled on the basis of the position of the catheter displayed on the X-ray image.

Referring to FIG. 9, in operation S901, a catheter is inserted into a blood vessel of a test object. When the catheter is inserted into the blood vessel, a dye is injected into the blood vessel through the catheter and an X-ray image for the blood vessel is captured in operation S902. The structure of the blood vessel may be ascertained from the captured X-ray image. In operation S903, a three-dimensional OCT image for the vicinity of the catheter is acquired while moving the catheter. In detail, an OCT image for a cross-section of the blood vessel in the thickness direction and an OCT image for a cross-section of the blood vessel in the longitudinal direction may be acquired. An example of the acquired three-dimensional OCT image is illustrated in FIG. 4.

When both the X-ray image and the three-dimensional OCT image are acquired, the shapes of the blood vessels respectively shown in the three-dimensional OCT image and the X-ray image are compared with each other to match the two images with each other in operation S904. Specifically, the corresponding portions thereof may be searched for using, for example, a method of comparing boundary patterns of the blood vessels shown in the two images with each other or a method of comparing characteristics of the blood vessel such as a gradient or the degree of bending of the blood vessel. When the two images are matched with each other, the position of the catheter in the three-dimensional OCT image is made to correspond to the blood vessel shown in the X-ray image to ascertain the position of the catheter in operation S905.

When the position of the catheter is ascertained, the ascertained position of the catheter is displayed on the X-ray image in operation S906. Finally, a subsequent moving route of the catheter is controlled on the basis of the position of the catheter displayed on the X-ray image.

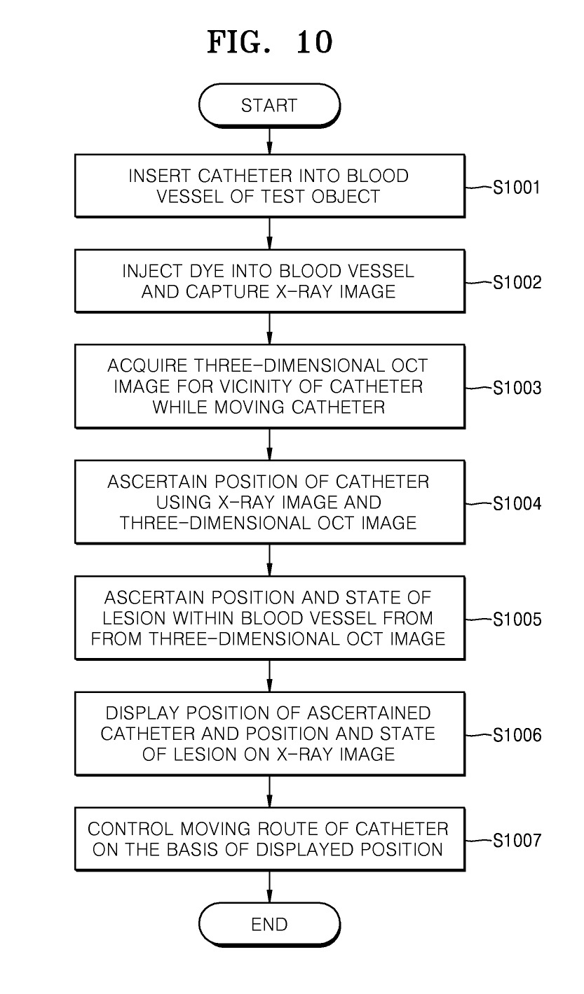

Referring to FIG. 10, a catheter is inserted into a blood vessel of a test object. When the catheter is inserted into the blood vessel, a dye is injected into the blood vessel through the catheter and an X-ray image for the blood vessel is captured in operation S1001. The structure of the blood vessel may be ascertained from the captured X-ray image. In operation S1003, a three-dimensional OCT image for the vicinity of the catheter is acquired while moving the catheter. In detail, an OCT image for a cross-section of the blood vessel in the thickness direction and an OCT image for a cross-section of the blood vessel in the longitudinal direction may be acquired. An example of the acquired three-dimensional OCT image is illustrated in FIG. 4.

When both the X-ray image and the three-dimensional OCT image are acquired, the position of the catheter is ascertained using the X-ray image and the three-dimensional OCT image in operation S1004. A description of the ascertainment of the position of the catheter using the two images is given above with reference to FIG. 9.

In operation S1005, the position and state of a lesion within the blood vessel is ascertained from the three-dimensional OCT image. Accordingly, an exact position and state of the lesion may be ascertained.

When the position of the catheter and the position and state of the lesion are ascertained, the ascertained content are displayed on the X-ray image in operation S1006. The position of the catheter may be displayed in a different color, and the position and state of the lesion may be displayed in various ways using characters or colors.

Finally, in operation S1007, a moving route of the catheter is controlled on the basis of the displayed position of the catheter.

As described above, according to the one or more of the above exemplary embodiments, a three-dimensional OCT image for the inside of a blood vessel in the vicinity of an angiocatheter is captured, and the captured three-dimensional OCT image is matched with an X-ray image for the blood vessel, thereby allowing the position of a catheter within the blood vessel to be ascertained.

Accordingly, X-ray imaging is performed on a region of interest (ROI) only once, a moving route of the catheter is controlled while capturing the three-dimensional OCT image, and then this may be displayed on the X-ray image.

In addition, an exact position of a lesion is ascertained using the captured three-dimensional OCT image, and the position may be displayed on the X-ray image.

Furthermore, positional information of the catheter and tissue information of the blood vessel are acquired in real time, and the moving route of the catheter may be controlled on the basis of the pieces of information, thereby preventing a wall or tissues of the blood vessel or tissues from being damaged.

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

What is claimed is:

1. A method of imaging a catheter using optical coherence tomography (OCT), the method comprising:
inserting a catheter into a blood vessel of a test object;
injecting a dye into the blood vessel and capturing an X-ray image;
acquiring a three-dimensional OCT image of a vicinity around the catheter;
determining a position of the catheter within the blood vessel using the three-dimensional OCT image and the X-ray image; and
displaying the position of the catheter on the X-ray image.

2. The method of claim 1, further comprising:
controlling a movement route through the blood vessel of the catheter on the basis of the displayed position.

3. The method of claim 1, wherein the determining of the position of the catheter comprises:
matching the three-dimensional OCT image and the X-ray image with each other by comparing shapes of the blood vessels shown in the three-dimensional OCT image with shapes of the blood vessels shown in the X-ray image, and
determining a position of the catheter in the blood vessel shown in the X-ray image based on the position of the catheter in the three-dimensional OCT image.

4. The method of claim 3, wherein the matching of the three-dimensional OCT image and the X-ray image comprises:
matching the three-dimensional OCT image and the X-ray image by comparing boundary patterns of the blood vessels shown in the three-dimensional OCT image and the X-ray image.

5. The method of claim 3, wherein the matching of the three-dimensional OCT image and the X-ray image comprises:
matching the three-dimensional OCT image and the X-ray image by comparing gradients and the degrees of bending of the blood vessels shown in the three-dimensional OCT image and the X-ray image.

6. The method of claim 4, wherein the matching of the three-dimensional OCT image and the X-ray image comprises:
matching the three-dimensional OCT image and the X-ray image by segmenting the blood vessels shown in the three-dimensional OCT image and the X-ray image into a plurality of portions and then comparing the segmented portions with each other.

7. The method of claim 5, wherein the matching of the three-dimensional OCT image and the X-ray image comprises:
matching the three-dimensional OCT image and the X-ray image by segmenting the blood vessels shown in the three-dimensional OCT image and the X-ray image into a plurality of portions and then comparing the segmented portions with each other.

8. The method of claim 3, wherein the three-dimensional OCT image is a blood vessel image constituted by at least one of a three-dimensional OCT image taken continuously over time and an OCT image showing a cross-section of the blood vessel image taken in one direction.

9. The method of claim 1, further comprising:
determining a position and a state of a lesion within the blood vessel using the three-dimensional OCT image; and
displaying the position and the state of the lesion on the X-ray image.

10. The method of claim 1, further comprising:
injecting a dye into a region of the blood vessel in which the catheter is placed and capturing another X-ray image in response to the catheter moving out of the region of the X-ray image.

11. The method of claim 1, further comprising:
measuring a movement distance and a movement time of the catheter; and
calculating a movement speed of the catheter based on the measured movement distance and movement time.

12. The method of claim 1, wherein the displaying of the position of the catheter comprises:
displaying the three-dimensional OCT image at the determined position of the catheter.

13. A non-transitory computer readable medium on which are stored computer instructions and data that, when executed by a computer, execute the method of claim 1.

14. An angiography apparatus comprising:
a catheter configured to be inserted into a blood vessel of a test object;
an optical probe configured to capture a three-dimensional optical coherence tomography (OCT) image of a vicinity around the catheter;
an X-ray image acquisition unit configured to acquire an X-ray image of the blood vessel of the test object into which a dye is injected;
a three-dimensional OCT image acquisition unit configured to receive the three-dimensional OCT image from the optical probe on the catheter;
a catheter position ascertainment unit configured to determine a position of the catheter within the blood vessel using the X-ray image acquired by the X-ray image acquisition unit and the three-dimensional OCT image acquired by the three-dimensional OCT image acquisition unit; and
an image display unit configured to display the ascertained position of the catheter on the X-ray image.

15. The angiography apparatus of claim 14, wherein the catheter position ascertainment unit comprises:
an image matching unit configured to match the three-dimensional OCT image and the X-ray image with each other by comparing shapes of the blood vessels shown in the three-dimensional OCT image with shapes of the blood vessels shown in the X-ray image, and
a position correspondence unit configured to determine a position of the catheter in the blood vessel shown in the X-ray image based on the position of the catheter in the three-dimensional OCT image.

16. The angiography apparatus of claim 15, wherein the image matching unit is further configured to match the three-dimensional OCT image and the X-ray image by comparing boundary patterns of the blood vessels shown in the three-dimensional OCT image and the X-ray image.

17. The angiography apparatus of claim 15, wherein the image matching unit is further configured to match the three-dimensional OCT image and the X-ray image by comparing gradients and the degrees of bending of the blood vessels shown in the three-dimensional OCT image and the X-ray image.

18. The angiography apparatus of claim 16, wherein the image matching unit is further configured to match the three-dimensional OCT image and the X-ray image by segmenting the blood vessels shown in the three-dimensional OCT image and the X-ray image into a plurality of portions and then compare the segmented portions with each other.

19. The angiography apparatus of claim 17, wherein the image matching unit is further configured to match the three-dimensional OCT image and the X-ray image by segmenting the blood vessels shown in the three-dimensional OCT image and the X-ray image into a plurality of portions and then compare the segmented portions with each other.

20. The angiography apparatus of claim 15, wherein the three-dimensional OCT image is a blood vessel image constituted by at least one of a three-dimensional OCT image taken continuously over time and an OCT image showing a cross-section of the blood vessel image taken in one direction.

21. The angiography apparatus of claim 14, further comprising:
an image analysis unit configured to determine a position and a state of a lesion within the blood vessel using the three-dimensional OCT image acquired by the three-dimensional OCT image acquisition unit,
wherein the image display unit is configured to display the position and the state of the lesion on the X-ray image.

22. The angiography apparatus of claim 14, wherein the X-ray image acquisition unit is further configured to acquire another X-ray image of the region in which the catheter is placed in response to the catheter moving out of a region of the X-ray image.

23. The angiography apparatus of claim 14, wherein the image display unit displays the three-dimensional OCT image received by the three-dimensional OCT image acquisition unit at the determined position of the catheter.

24. A medical imagining device comprising:
a three-dimensional (3D) optical coherence tomography (OCT) image acquisition unit configured to receive a 3D OCT image from an optical probe;
an X-ray image acquisition unit configured to receive an X-ray image; and
a catheter position ascertainment unit configured to determine a position of a catheter by matching the 3D OCT image and the X-ray image.

25. The medical imaging device of claim 24, further comprising:
an image analysis unit configured to analyze the 3D OCT image to ascertain a position and a state of a lesion within a blood vessel, and ascertain the structure of the blood vessel.

26. A method of controlling a medical imagining device, the method comprising:
receiving, at a three-dimensional (3D) optical coherence tomography (OCT) image acquisition unit, a 3D OCT image from an optical probe;
receiving, at an X-ray image acquisition unit, an X-ray image; and
determining, using a catheter position ascertainment unit, a position of a catheter by matching the 3D OCT image and the X-ray image.

27. The method of claim 26, further comprising:
analyzing, using an image analysis unit, the 3D OCT image to ascertain a position and a state of a lesion within a blood vessel and ascertain the structure of the blood vessel.
